(19) United States
(12) Patent Application Publication
(10) Pub. No.: US 2017/0136136 A1
(43) Pub. Date: May 18, 2017

(54) ON-DEMAND MEDICAL DEVICE STERILIZATION DEVICE AND METHODS

(71) Applicant: Rayvio Corporation, Hayward, CA (US)

(72) Inventors: Sheung LI, Hayward, CA (US); Doug COLLINS, Hayward, CA (US); Yitao LIAO, Hayward, CA (US)

(21) Appl. No.: 15/352,280

(22) Filed: Nov. 15, 2016

Related U.S. Application Data
(60) Provisional application No. 62/256,003, filed on Nov. 16, 2015.

(51) Int. Cl. A61L 2/10; A61L 2/24

(52) U.S. Cl. CPC A61L 2/10; A61L 2/24; A61L 2202/24; A61L 2202/14

(57) ABSTRACT

A portable medical sanitation device includes a power source, a solid-state UV light source for outputting UV light (e.g. within the UV-C band), a controller for directing the plurality of solid-state UV light sources to output UV light, a sterilization chamber comprising a cavity having side-walls having the solid-state UV light source coupled thereto, wherein the sterilization chamber is configured to receive UV light from the plurality of solid-state UV light sources, and wherein the side-walls are configured to reflect UV light incident thereto, and a housing configured to contain the power source, the plurality of solid-state UV light sources, the controller and the sterilization chamber.

### CROSS-REFERENCE TO RELATED CASES

The present invention is a non-provisional of and claims priority to U.S. Application No. 62/256,003 filed Nov. 16, 2015, incorporated by reference herein, for all purposes.

### BACKGROUND OF THE INVENTION

The present invention relates to the field of medical device sanitation. More specifically, the present invention relates to a just-in-time or on-demand medical device sanitation devices and methods.

Previously, reusable medical equipment are subject to sanitation regiments to reduce cross-contamination of diseases or pathogens from one patient to another patient. Sanitation regiments for metallic equipment, e.g. scalpels, drills, etc. typically include high-temperature steam-baking (autoclaving) procedures; whereas for soft goods, e.g. catheters, probes, etc. typically include alcohol or other sanitizing material wipe down procedures.

The inventors of the present invention have determined that there are potential problems with typical hospital sanitation regiments. More specifically, after sanitation, the medical device may sit on a shelf for months before it is used. In that state, germs, viruses, and other pathogens that have not completely been removed by the sanitation regiments may spread; pathogens may be airborne and land upon the equipment; and the like. In such situations, the stored medical equipment is used on patients without further sanitation.

In light of the above, what is desired are methods and devices for medical device sanitation without the drawbacks discussed above.

### SUMMARY

The present invention relates to the field of medical device sanitation. More specifically, the present invention relates to a medical device sanitation devices and methods.

Embodiments of the present invention include a portable medical sanitation device that can sterilize medical devices just before they are used, or shortly before the devices are used. Embodiments include a portable device including a UV sterilization chamber. Medical devices can be placed in the UV sterilization chamber and exposed to UV light within the UV-C band, before the devices are to be used. Various embodiments use proprietary UV-C LEDs thus UV exposure time is typically short, e.g. less than 30 seconds, depending upon the intensity of UV light provided, thus the sterilization is considered just-in-time or on-demand.

In various embodiments, solid-state UV light sources, e.g. UV-LEDs, are used to provide the UV light. The inventors believe that there are substantial advantages of using solid-state device, as compared to mercury tube UV light sources. Some advantages include that solid-state devices have much lower power requirements, so that the sanitation devices may reliably run on a battery for substantial periods of time. This greatly increases the portability, flexibility and convenience of such sanitation devices. Other advantages include that longevity of solid-state UV light sources is believed to be much greater than UV mercury lights as mercury lights typically use fragile glass tubes, and as repeated power on/off cycles are known to reduce the longevity of the mercury bulb. Still other advantages includes that solid-state UV light sources do not include hazardous materials such as mercury and breakage of solid-state UV LEDs do not poison workers and do not contaminate the working environment. The inventors consider such advantages particularly important within the health care environment.

In one aspect of the invention, a portable medical sanitation device is disclosed. One apparatus includes a power source, and a plurality of solid-state UV light sources coupled to the power source, wherein the plurality of solid-state UV light sources are configured to output UV light within the UV-C frequency band. A device may include a controller coupled to the power source and to the plurality of solid-state UV light sources, wherein the controller is configured to direct the plurality of solid-state UV light sources to output UV light, and a sterilization chamber coupled to the plurality of solid-state UV light sources, wherein the sterilization chamber comprises a cavity having side-walls, wherein the sterilization chamber is configured to receive UV light within the UV-C frequency band from the plurality of solid-state UV light sources, and wherein the side-walls are configured to reflect or disperse UV light incident thereto. In some systems, a housing is configured to contain the power source, the plurality of solid-state UV light sources, the controller and the sterilization chamber.

### BRIEF DESCRIPTION OF THE DRAWINGS

In order to more fully understand the present invention, reference is made to the accompanying drawings. Understanding that these drawings are not to be considered limitations in the scope of the invention, the presently described embodiments and the presently understood best mode of the invention are described with additional detail through use of the accompanying drawings in which:

FIG. 1 illustrates a block diagram of embodiments of the present invention; and

FIGS. 2A-2B illustrate cross-sections of various embodiments of the present invention.

### DETAILED DESCRIPTION

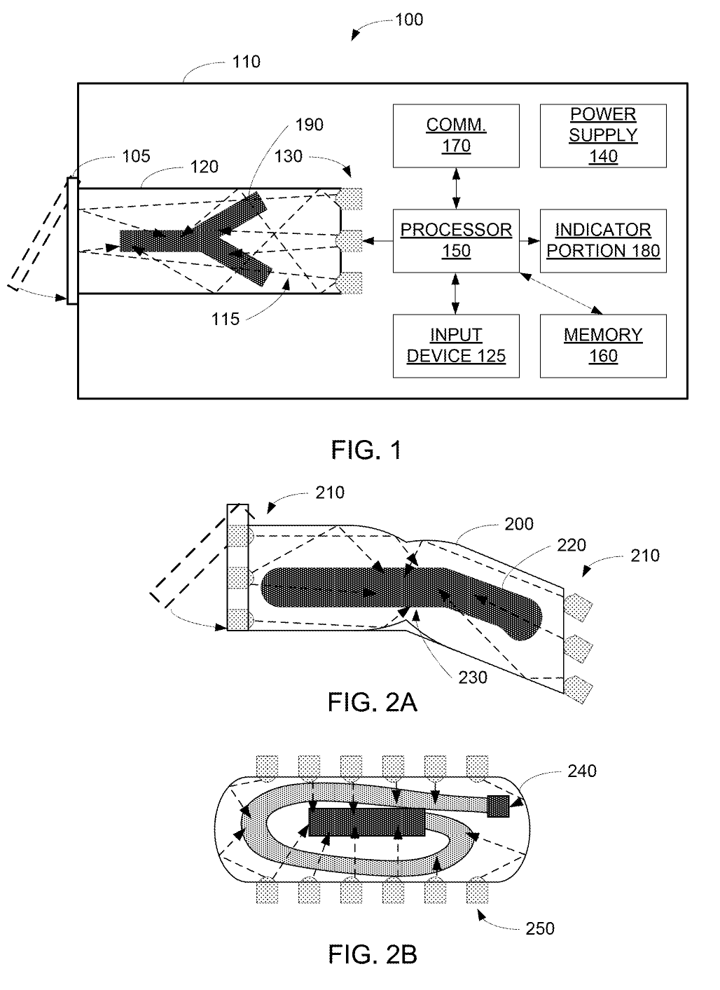

FIG. 1 illustrates a block diagram of various embodiments of the present invention. In FIG. 1, a sterilization device 100 is disclosed including a housing 110, a sterilization chamber 120, a plurality of UV LEDs 130, a power supply 140, a controller 150, a memory 160, a communications portion 170, and a user indicator 180. As can be seen, a door 105 is illustrated that closes an opening of sterilization chamber 120. Inside sterilization chamber 120 a medical device 190 is illustrated receiving UV-C light 115.

In various embodiments, sterilization device 100 is a hand-held, portable device that includes its own power supply 140, e.g. batteries (e.g. rechargeable battery). Within sanitation device 100, processor 150 controls the sterilization process, storage of data, and other operations of device, as will be described-below. As shown, device 100 includes multiple solid-state UV-C light sources 130, e.g. LEDs, that provide UV-C light 115. In various embodiments, solid-state UV-C light sources, under development by the assignee of the present application, are used. Accordingly, the sterilization process is very quick, e.g. <2 minutes, <1 minute, <30 seconds, or the like, and can be performed immediately before the medical device is required to be used.

In various embodiments, sterilization chamber 120 may be constructed of one or more materials having an interior surface that reflects and/or disperses UV-C light. In some examples, chamber 120 may include a shiny or textured material such as: aluminum, stainless steel, silver, Teflon, or the like. In other examples, chamber 120 may include an outer base surface material made of plastic, glass, metal, or the like and have an interior coating of Teflon, silver, aluminum or other UV reflective or dispersive material. In various embodiments, sterilization chamber 120 may be approximately cylindrical (e.g. circular, ovoid); approximately prismatic (e.g. triangular, rectangular, quadrilateral, hexagonal); or any other shape. Additionally, the bases or side-walls of chamber 120 need not be parallel in some embodiments.

In some embodiments, an input device 125 may be provided to capture an identifier of the medical device and/or an identifier of the user of the sterilization device. In some examples, reader 125 may optically capture two or three dimensional bar codes, may sense an RFID tag, capture data via an NFC tag, capture GPS signals, capture Wi-Fi or other beacon signals, or the like. In other examples, input device 125 may also or alternatively include a soft or physical alphanumeric keyboard for the user to type in one or more sets of data that are used to identify the medical device being sterilized and/or the identity of the user, or the like.

In various embodiments, memory 160 may be used to store one or more sterilization routines that are executable on processor 150. For example, one sterilization routine may be provided for clear plastic devices that specifies 30 seconds of UV light; another sterilization routine may be provided for opaque plastic devices that specifies 120 seconds of UV light; still other sterilization routines may be provided for tubing of different composition; and the like. Additionally, memory 160 may be used to store usage data, indicating when a sterilization process is initiated. In some examples, the identification data captured by input device 125 (e.g. identifier of the device being sanitized; identifier of the person performing the request; identifier of the geographic location or logical location (e.g. room number or station of the health-care facility) where the device is sanitized; time; and the like may also be stored in memory 160. These data together may be stored in a data log for evidentiary/certification purposes, for example, to certify that patient-safety best-practices are followed within a hospital.

In FIG. 1, a communications portion 170 is provided to support wired and/or wireless communication between sterilization device 100 and a remote device, e.g. a hospital database server, a smart device (e.g. iPhone), or the like. Data transferred may include data from memory 160, including any of the usage data discussed above, firmware data, and the like. In various embodiments, wired connections may include: USB, Firewire, Apple Lightning, or the like. In addition or alternatively, embodiments may include wireless communication mechanisms, such as Bluetooth, Wi-Fi, NFC, Zigbee, Zwave, cellular data (e.g. 4G, LTE, 3G), IR, or the like.

In some embodiments, an indicator portion 180 may include one or more indicator lights (e.g. LED), a display, a speaker, a vibrating device, or the like. Indicator portion 180 may be used to provide a status to the user, such as: when a sterilization process is being performed; when the sterilization process is finished; if the sterilization process is not complete; when sterilization device 100 is on; when sterilization device 100 is transmitting data, and the like.

Embodiments that have additional or less functionality are contemplated. For example, in one embodiment, sterilization device 100 may include a housing 110, a power supply, 140, a sterilization chamber 120, and a plurality of UV LEDs 130. Such embodiments may be paired with and controlled by the smart device. For example, using an App on an iPhone, a user may direct sterilization device 100 to begin a UV sanitation/exposure cycle (e.g. directly via the app or via a wireless messaging technique, e.g. a text message). Next, in response sterilization device 100 runs the sterilization routine (e.g. determine the device being sanitized, determine the settings, run the sanitation routine), and then the sterilization device 100 indicates (e.g. directly via the app or a wireless messaging technique, e.g. a return text message) to the iPhone that the sterilization routine is finished and the medical device is ready to use.

FIGS. 2A-B illustrate various embodiments of the present invention. In the example in FIG. 2A, chamber 200 is illustrated including UV LEDs 210 disposed on one or both of the bases of chamber 200. In various examples, the walls of chamber 200 need not be uniform, parallel or straight, or the like, and may vary according to the shape of the device 220 being sanitized. As illustrated, the walls of chamber 200 may help concentrate or disperse the sanitizing UV light 230 as required. In some embodiments, the end walls of chamber 200 need not be parallel or planar, and may also be shaped to help uniformly disperse UV light 230.

In the example in FIG. 2B, a sterilization chamber 240 may be approximately cylindrical in shape and include UV LEDs 250 along the sidewalls of chamber 240. In this embodiment, the end portions need not be flat or parallel. In other embodiments, UV LEDs 250 may be distributed via optics, e.g. fiber optics, optical plates, or the like, into sterilization chamber 240, thus relying upon total internal reflection of the UV illumination within the optics.

In light of the present disclosure, it is believed that one of ordinary skill in the art will recognize additional configurations for a sterilization chamber that can be implemented.

In various embodiments, a sterilization process may include the following steps:

1. Receiving a medical device to sterilize in a sterilization device;

2. determining automatically or manually, an identification of the medical device;

3. determining automatically or manually, an identification of a user, and the device and user identifications in memory;

4. the processor determining that the sterilization chamber is closed, secured, or otherwise enclosed, and the user (via direct input or via app) initiating the sanitation process:

5. illuminating the UV-LEDs under direction of the processor, to provide the UV-C upon the medical device (via direct or reflection); and

6 turning off the UV-LEDs off under direction of the processor when a desired amount of time has elapsed, and/or the sanitation process is stopped;

7. Storing the completion data in the memory under direction of the processor;

8. Subsequently, transmitting the identifier of the medical device, the user, the time, indication of completion of the sanitation cycle, and the like from the memory via the communications path to a remote device (e.g. hospital server, compliance server, or the like).

In various embodiments, the sterilization chamber may be physically adapted for specific medical devices, such as an intravenous hub, a catheter, an endoscope, an ultrasonic probe, thermometer, a CPAP-type device, fluid switches, adult personal toys, or the like.

In other embodiments, combinations or sub-combinations of the above disclosed invention can be advantageously made. For example, in some embodiments, the position of an opening and of a door may be situated on other portions of a sterilization chamber such as along the length-wise direction of the sterilization chamber. In some embodiments, one or more supporting structures may be provided to help suspend the object to be sanitized away from the walls of sterilization chamber. Some examples may include a metalized wire rack, transparent plastic rack, a series of thin metal (e.g. plano wire) or plastic wires (e.g. fishing line) may crisscross the sterilization chamber suspend the object, or the like. In another example, the sterilization chamber may be embodied as a drawer-type unit in a cabinet and UV LEDs may disposed on the inside of a drawer. In operation, after the drawer is pushed into the cabinet, the UV LEDs may be activated. In additional embodiments, one or more photodiodes may be included that can be exposed to the UV light from the UV LEDs. In operation, when UV light is present in the chamber, the photodiodes detect the UV light, and an indicator light may be activated on the exterior of the device, a sound may be played, or the like.

Some of the embodiments described above may include a number of other capabilities, such as a portion that provides a liquid or mist immersion of the device by a disinfection liquid (e.g. alcohol), followed by or preceded by a UV-light exposure cycle. In other embodiments, heat (e.g. IR LED, heat lamp, etc.) and/or physical agitation may also be used. In some embodiments, the side-walls of the sanitizing chamber may include a UV-reactive material, such as TiO2, or the like. In such embodiments, when UV strikes the coating, active metal species of oxygen or water may be generated which in turn, react with contaminants on the surface of the chamber. The block diagrams of the architecture and flow charts are grouped for ease of understanding. However it should be understood that combinations of blocks, additions of new blocks, re-arrangement of blocks, and the like are contemplated in alternative embodiments of the present invention.

The specification and drawings are, accordingly, to be regarded in an illustrative rather than a restrictive sense. It will, however, be evident that various modifications and changes may be made thereunto without departing from the broader spirit and scope of the invention as set forth in the claims.

What is claimed is:

1. A portable medical sanitation device comprising:
a power source;
a plurality of solid-state UV light sources coupled to the power source, wherein the plurality of solid-state UV light sources are configured to output UV light within the UV-C frequency band;
a controller coupled to the power source and to the plurality of solid-state UV light sources, wherein the controller is configured to direct the plurality of solid-state UV light sources to output UV light;
a sterilization chamber coupled to the plurality of solid-state UV light sources, wherein the sterilization chamber comprises a cavity having side-walls, wherein the sterilization chamber is configured to receive UV light within the UV-C frequency band from the plurality of solid-state UV light sources, and wherein the side-walls are configured to reflect UV light incident thereto; and
a housing configured to contain the power source, the plurality of solid-state UV light sources, the controller and the sterilization chamber.

2. The device of claim 1 further comprising:
a memory coupled to the controller, wherein the memory is configured to store an sanitation time or an exposure duration associated with the plurality of solid-state UV light sources outputting UV light;
wherein the controller is configured to determine the sanitation time or the exposure duration associated with the plurality of solid-state UV light sources outputting UV light.

3. The device of claim 2 further comprising:
a communication portion coupled to the memory, wherein the communication portion is configured to provide the sanitation time or the exposure duration to a remote server; and
wherein the controller is configured to direct the communication portion to communicate with the remote server.

4. The device of claim 2 further comprising:
an identification reader coupled to the controller, wherein the identification reader is configured to obtain identification data of a medical device disposed within the sterilization chamber;
wherein the controller is configured to direct the identification reader to obtain the identification data; and
wherein the memory is configured to store the identification data of the medical device.

5. The device of claim 1 wherein the housing comprises a closable opening, wherein the closable opening is configured to allow a user to place a medical device within the sterilization chamber.

6. The device of claim 5 wherein the medical device is selected from a group consisting of: an intravenous hub, a catheter, an endoscope, and an ultrasonic probe.

7. The device of claim 5 wherein at least some solid-state UV light sources from the plurality of solid-state UV devices are disposed upon the closable opening.

8. The device of claim 5 wherein at least some solid-state UV light sources from the plurality of solid-state UV devices are disposed along the sidewalls of the sanitation chamber.

9. The device of claim 1 wherein the sterilization chamber comprises an approximately cylindrical tube, wherein at least a first group of solid-state UV light sources are disposed upon a first base of the approximately cylindrical tube, and wherein at least a second group of solid-state UV light sources are disposed upon a second base of the approximately cylindrical tube.

10. The device of claim 1 wherein the sidewalls of the sterilization chamber are shaped such that some UV light from the UV light from the plurality of solid-state UV light sources are approximately focused within a first volumetric region within the sterilization chamber.

11. The device of claim 1
wherein the sterilization chamber comprises a support material and a UV reflective coating material;
wherein the UV reflective coating material is selected from a group consisting of: aluminum, Teflon, and
wherein the support material is selected from a group consisting of: plastic, metal, glass.

12. A method for a portable medical sanitation device comprising:
receiving a medical device within a sterilization chamber of the portable medical sanitation device;
determining with a controller, whether the sterilization chamber is secured;
providing with a plurality of solid-state UV light sources, under direction of the controller, UV light within the UV-C frequency band to the medical device disposed within the sterilization chamber in response to a determination that the sterilization chamber is secured;
determining a sterilized condition by the controller, when the medical device has been provided with a requisite amount of the UV light;
indicating sterilization completion to a user, under direction of the controller, in response to the sterilized condition being determined; and
ensuring with the controller, that when the medical device is removed from the sterilization chamber, the plurality of solid-state UV light sources do not provide the UV light.

13. The method of claim 12 further comprising:
storing under direction of the controller, a sanitation time or an exposure duration associated with the plurality of solid-state UV light sources providing UV light to the medical device in a memory, in response to the sterilized condition being determined.

14. The method of claim 12 further comprising:
providing, with a communication portion, the sanitation time or the exposure duration to a remote server in response to the sterilized condition being determined.

15. The method of claim 13 further comprising:
determining, with an identification reader, identification data of the medical device disposed within the sterilization chamber; and
providing, with the communication portion, the identification data of the medical device to the remote server in response to the sterilized condition being determined.

16. The method of claim 12 wherein the determining with the controller, whether the sterilization chamber is secured comprises determining, with the controller, whether
a closable opening of the device is closed.

17. The method of claim 16 wherein the medical device is selected from a group consisting of: an intravenous hub, a catheter, an endoscope, and an ultrasonic probe.

18. The method of claim 16 wherein the providing with the plurality of solid-state UV light sources comprises: providing UV light within the UV-C frequency band from sidewalls of the sterilization chamber.

19. The method of claim 18 wherein at least some the UV light reflects from surfaces of the sidewalls of the sterilization chamber.

20. The method of claim 19 wherein the some UV light that reflects from the surfaces of the sidewalls are approximately concentrated within a first volumetric region within the sterilization chamber.
